(19) United States
(12) Patent Application Publication
(10) Pub. No.: US 2018/0036022 A1
(43) Pub. Date: Feb. 8, 2018

(54) DEVICE FOR IMPROVING ELECTROHYDRAULIC LITHOTRIPSY PROBE STIFFNESS

(71) Applicant: COOK MEDICAL TECHNOLOGIES LLC, Bloomington, IN (US)

(72) Inventors: Michael Lee Williams, Pinnacle, NC (US); Kenneth C. Kennedy, Clemmons, NC (US)

(73) Assignee: Cook Medical Technologies LLC

(21) Appl. No.: 15/664,095

(22) Filed: Jul. 31, 2017

Related U.S. Application Data
(60) Provisional application No. 62/371,437, filed on Aug. 5, 2016.

(51) Int. Cl. A61B 17/22; A61M 25/00; A61B 17/00

(52) U.S. Cl. CPC A61B 17/22012; A61B 17/00234; A61M 25/0053; A61B 2017/0034; A61B 2017/22025

(57) ABSTRACT

A lithotripsy probe is provided including an elongate body with a proximal end, a distal end, and a lumen extending therethrough. The lithotripsy probe further includes a stiffening element with a proximal end, a distal end, and a length extending from the proximal end to the distal end. The stiffening element is disposed within the lumen of the elongate body. The stiffening element has a stiffness that varies along the length of the stiffening element, where the stiffness of the proximal end of the stiffening element is greater than the stiffness of the distal end of the stiffening element.

### CROSS-REFERENCE TO RELATED APPLICATIONS

The present patent document claims the benefit of the filing date under 35 U.S.C. §119(e) of Provisional U.S. Patent Application Ser. No. 62/371,437 filed Aug. 5, 2016, which is hereby incorporated by reference.

### FIELD

The present disclosure relates to medical devices and more specifically to electrohydraulic lithotripsy probes.

### BACKGROUND

The statements in this section merely provide background information related to the present disclosure and may not constitute prior art.

Electrohydraulic lithotripsy is a procedure used as a means to break up stones within the biliary tree and urinary tract. While many stones may naturally pass through and out of the patient, some stones are too large to be passed on their own. These stones may become stuck in the biliary tree or urinary tract, thereby requiring medical intervention. A common way to remove stones is with lithotripsy: a medical procedure that involves breaking up the stones into smaller pieces that are then able to be passed naturally out of the patient's body. One specific subset of lithotripsy is electrohydraulic lithotripsy, which employs high energy shock waves to fragment the stones. These shock waves can be generated and targeted at the stone from outside of the patient's body or with a device that is inserted into the patient's body—either percutaneously or through a natural body cavity.

Electrohydraulic lithotripsy can use a shock wave generating device that is inserted into the patient's body. The device, or probe, is most commonly passed through an accessory channel of a scope or other similar introducer device until the probe is adjacent to the stone. A shock wave is then generated through the probe towards the stone. Eventually, the shock waves cause the stone to fragment and the probe and scope may then be removed while the stone fragments naturally pass through and out of the patient's body. Alternatively, the fragments may be removed by a vacuum, basket, or other fragment collection device inserted through or with the scope.

The scope, which is often a cholangioscope, must have an outer diameter small enough to allow it to be safely advanced through a body lumen of a patient. Sometimes, the cholangioscope is advanced through a working channel of a larger duodenoscope that also must have a diameter small enough to allow it be safely advanced through a body lumen of a patient. Since the probe is passed through a working channel of one of these scopes, the outer diameter of the probe must be fairly small. However, these probes are generally quite long, with lengths often exceeding 230 centimeters. Because of the high length to diameter ratio, one common problem associated with electrohydraulic lithotripsy is the buckling or kinking of the probe as it is advanced through the working channel of the scope and into a patient's body lumen. Kinking and buckling of the probe can be caused by the friction generated between the probe and the working channel of the scope or various structures in the patient's body lumen. As the physician advances the probe further into the scope, the friction between the scope and probe increase, thus requiring a greater force to further advance the probe. However, as the physician applies more force to the proximal end of the probe, the probe is more likely to kink or buckle, as it cannot withstand a large force due to its small diameter and low strength. When the probe kinks or buckles, the physician may have increased difficulty in advancing the probe towards the stone. Additionally, the probe must also maintain sufficient flexibility as it must be navigated through the twists and turns of the patient's body lumen.

Thus, it is desirable to provide a lithotripsy probe that is resistant to kinking and buckling while maintaining a small outer diameter with sufficient flexibility that may be passed through the working channel of a scope.

### SUMMARY

In one form of the present disclosure, a lithotripsy probe is provided. The lithotripsy probe comprises an elongate body comprising a proximal end, a distal end, and a lumen extending therethrough. The lithotripsy probe also comprises a stiffening element comprising a proximal end, a distal end, and a length extending from the proximal end to the distal end. The stiffening element is disposed within the lumen of the elongate body. Further, the stiffening element comprises a stiffness that varies along the length of the stiffening element, wherein the stiffness of the proximal end of the stiffening element is greater than the stiffness of the distal end of the stiffening element.

The lithotripsy probe may further comprise first and second conductive wires extending through the lumen of the elongate body, the first and second conductive wires configured to deliver electrical energy to the distal end of the elongate body. Also, the stiffening element of the lithotripsy probe may increase along the length of the stiffening element from a lower stiffness at the distal end of the stiffening element to a greater stiffness at the proximal end of the stiffening element. Additionally, the proximal end of the stiffening element may be substantially coterminous with the proximal end of the elongate body and the distal end of the stiffening element may be substantially coterminous with the distal end of the elongate body. The stiffening element may also comprise a proximal portion and a distal portion, the proximal portion comprising a substantially constant outer diameter and the distal portion comprising a substantially constant outer diameter that is smaller than the outer diameter of the proximal portion, the stiffening element further comprising a step at a transition point between the proximal and distal portions. The stiffening element may include a proximal portion, a distal portion, and a first and second stiffener, the first stiffener extending from the proximal end of the stiffening element to the distal end of the stiffening element, the second stiffener extending through the proximal portion of the stiffening element. The stiffening element could also comprise a proximal portion comprising a first material and a distal portion comprising a second material, the first material having a greater stiffness than the second material. Alternatively, the stiffening element may comprise a proximal portion and a distal portion, the proximal portion being heat treated to increase the stiffness of the proximal portion, the proximal portion having a stiffness that is greater than the stiffness of the distal portion.

In another form of the present disclosure, a lithotripsy kit is provided. The lithotripsy kit comprises a scope comprising a proximal end, a distal end, and a working channel extending therethrough, the scope further comprising a proximal entrance to the working channel. The lithotripsy kit further comprises a probe comprising an elongate body comprising a proximal end, a distal end, and a lumen extending therethrough, the probe further comprising a stiffening element comprising a proximal end, a distal end, and a length extending from the proximal end to the distal end, the stiffening element disposed within the lumen of the elongate body. The probe is advancable through the working channel of the scope, the stiffening element comprising an adjacent point that is adjacent to the proximal entrance of the working channel, the adjacent point varying in position along the length of the stiffening element as the probe is distally advanced through the working channel, the probe further comprising a force required to advance the probe through the working channel of the scope, the force increasing as the adjacent point moves proximally along the length of the stiffening element. Also, the stiffening element further comprises a stiffness that varies along the length of the stiffening element, the stiffness increasing along the length of the stiffening element from the distal end to the proximal end, wherein the stiffness of the stiffening element at the adjacent point is proportional to the instant force required to advance the probe through the working channel of the scope at the adjacent point's current position along the length of the stiffening element.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a drawing of a lithotripsy probe, duodenoscope, and cholangioscope inserted into a patient's duodenum in accordance with the teachings of the present disclosure;

FIG. 2 is a drawing of a lithotripsy probe, duodenoscope, and cholangioscope inserted into a patient's duodenum;

FIG. 3 is a graph showing the relationship between the force required to advance the probe distally and the position of the distal end of the probe;

FIG. 4 is a cross-sectional view of a lithotripsy probe with a stiffening element;

FIG. 5 is an embodiment of a stiffening element with a varying diameter;

FIG. 6A is an embodiment of a stiffening element with a tapered distal portion;

FIG. 6B is an embodiment of a stiffening element with a stepped distal portion;

FIG. 6C is an embodiment of a stiffening element with a tapered central portion and tapered distal portion;

FIG. 6D is an embodiment of a stiffening element with a stepped central portion and a stepped distal portion;

FIG. 7A is an embodiment of a stiffening element with two stiffeners;

FIG. 7B is an embodiment of a stiffening element with three stiffeners;

FIG. 7C is an embodiment of a stiffening element with two stiffeners with tapers;

FIG. 8A is an embodiment of a stiffening element made up of two materials;

FIG. 8B is an embodiment of a stiffening element with a coated proximal portion; and

FIG. 8C is an embodiment of a stiffening element with heat treated portions.

### DETAILED DESCRIPTION

The following description is merely exemplary in nature and is not intended to limit the present disclosure, application, or uses. It should be understood that throughout the drawings, corresponding reference numerals indicate like or corresponding parts and features. It should also be understood that various cross-hatching patterns used in the drawings are not intended to limit the specific materials that may be employed with the present disclosure. The cross-hatching patterns are merely exemplary of preferable materials or are used to distinguish between adjacent or mating components illustrated within the drawings for purposes of clarity.

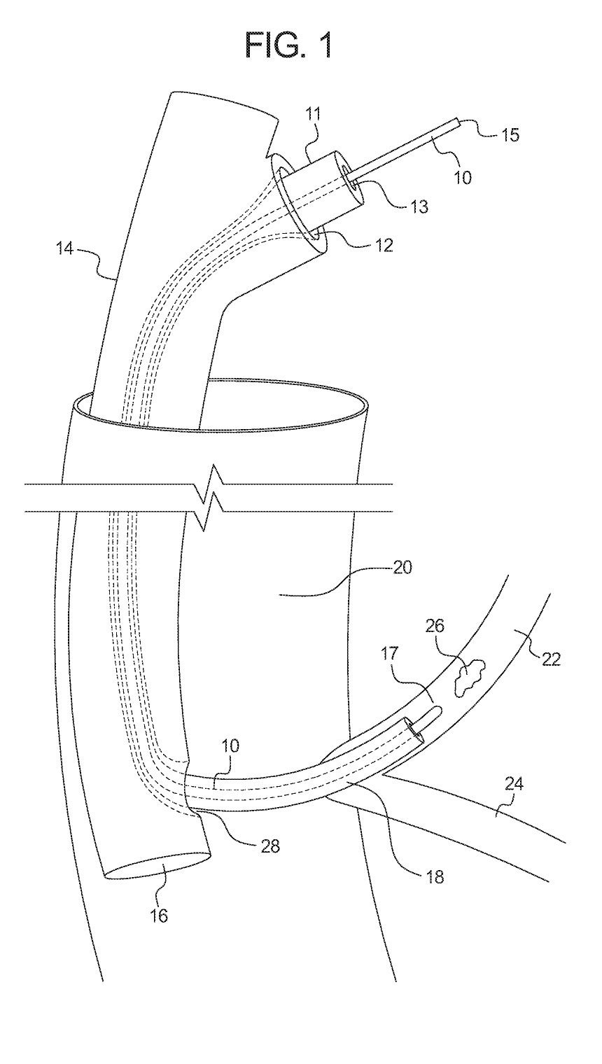

FIG. 1 shows a lithotripsy probe 10 with a proximal end 15 and a distal end 17 inserted into a working channel 13 of a cholangioscope 11, which is in turn inserted into a working channel 12 of a duodenoscope 14. In this example, the duodenoscope 14 is inserted into the mouth of a patient and through the digestive track until the distal end 16 of the duodenoscope 14 is near the papilla of Vater 18 in the duodenum 20. The papilla of Vater 18 is a mound-like structure that extends into the duodenum 20 and serves as the exit point for the common bile duct 22 and pancreatic duct 24. A stone 26 may be lodged in the common bile duct 22, and thus the probe 10 must be inserted through the papilla of Vater 18 until the distal end 17 of the probe 10 is near the stone 26. Before the probe 10 is inserted, the cholangioscope 11 may be inserted through the working channel 12 of the duodenoscope 14 and then pushed through the papilla of Vater 18 until the distal end of the cholangioscope 11 is adjacent to the stone 26. The probe 10 may then be inserted through a working channel 13 of the cholangioscope 11 until the distal end 17 of the probe 10 is near the stone 26. Alternatively, the probe 10 can be at least partially preloaded into the working channel 13 of the cholangioscope 11 and the probe 10 and cholangioscope 11 can be advanced through the working channel 12 of the duodenoscope 14 together. Once the distal end 17 of the probe 10 is near the stone 26, shock wave energy is applied through the probe 10 and towards the stone 26 which causes the stone 26 to fragment. The fragments of the stone 26 may either be collected and removed from the patient, or allowed to naturally pass through the patient's body. The probe 10, cholangioscope 11, and duodenoscope 14 may then be withdrawn from the patient's body, together or separately.

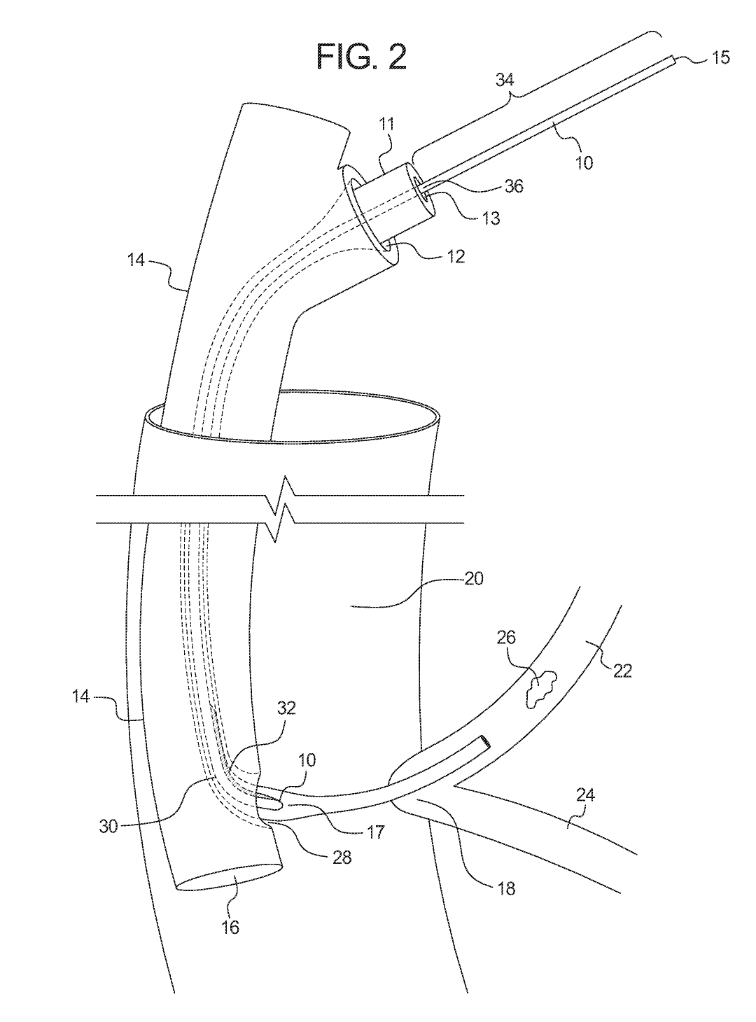

As the probe 10 is advanced through the working channel 13 of the cholangioscope 11, the frictional resistance between the working channel 13 and the probe 10 steadily increases the further the probe 10 is advanced due to the increasing amount of surface area contact between the probe 10 and working channel 13. Therefore, as the probe 10 is advanced the physician must apply an increasingly larger force to the proximal end of the probe 10 to overcome the increasing frictional forces. Due to the increasingly larger force that the physician must apply, the probe 10 may be at risk of kinking or buckling throughout this procedure. Further, the probe 10 is at a significant risk of buckling when the distal end 17 of the probe 10 reaches and then extends past the side port 28 of the duodenoscope 14 (FIG. 2). The cholangioscope 11 and the probe 10 are deflected near the side port 28 by an elevator (not shown) within the duodenoscope 14. The elevator can be manipulated by the physician to control the deflection of the cholangioscope 11 and probe 10 and thus steer the cholangioscope 11 and probe 10 towards the papilla of Vater 18 or other body structure. The elevator may deflect the cholangioscope 11 and probe 10 at this deflection point 30 as much as or more than 90 degrees. When the distal end 17 of the probe 10 reaches the elevator, the force necessary to further advance the probe 10 through the cholangioscope 11 increases due to the high amount of friction between the working channel 13 of the cholangioscope 11 and the probe 10 at the deflection point 30. Thus, the physician must apply a greater force to the proximal end of the probe 10 to continue to advance the probe 10 than was previously necessary.

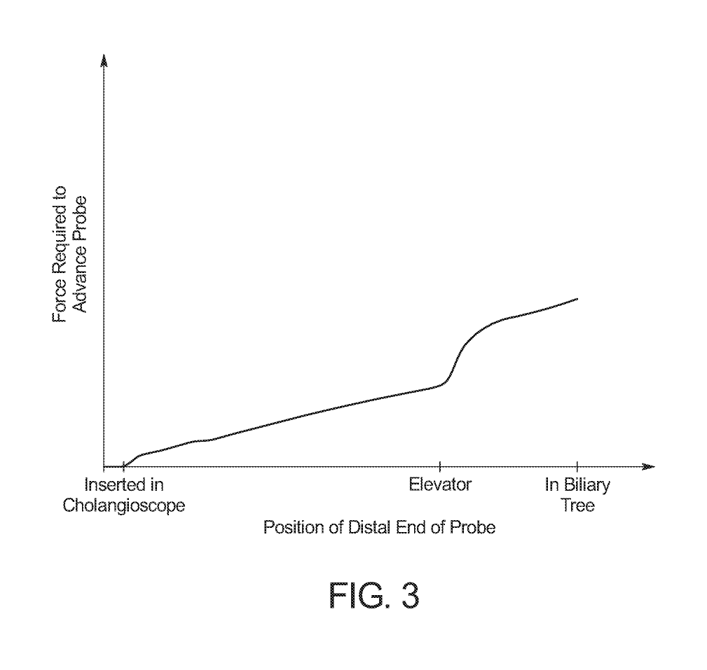

When all or part of the probe 10 is disposed within the working channel 13, the portion of the probe 10 that is within the working channel 13 may be less prone to kinking or buckling due to the additional support provided by the low tolerance between the working channel 13 and the probe 10. However, at any point in the procedure, an exposed portion 34 of the probe 10 (which varies in length based on the position of the probe 10 with respect to the cholangioscope 11) that has not yet been advanced into the working channel 13 of the cholangioscope 11 does not have the benefit of the support provided by the working channel 13. Thus, when the physician applies an increased force to the exposed portion 34 of the probe 10 to advance the probe 10 past the elevator and deflection point 30, the exposed portion 34 is prone to buckling, especially immediately proximal to the proximal entrance 36 to the working channel 13. A rough graph showing the relationship between the force required by the physician to distally advance the probe 10 and the position of the distal end 17 of the probe 10 is shown in FIG. 3.

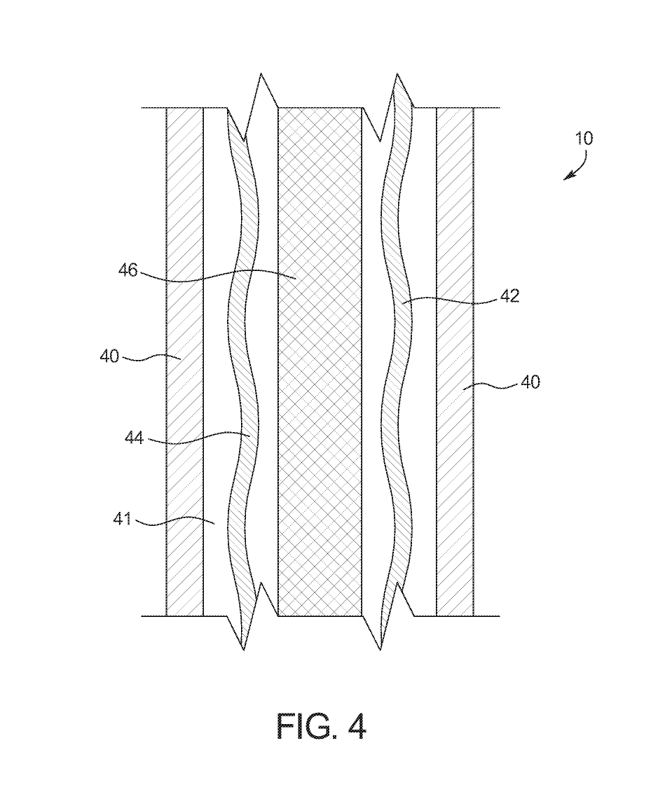

To help minimize the risk of buckling, a stiffening element 46 may be used with the probe 10. As shown in FIG. 4, the lithotripsy probe 10 may include a flexible tubular body 40 with a lumen 41 running therethrough. The flexible tubular body 40 may be made of a variety of biocompatible materials, including but not limited to: polytetrafluoroethylene. Two conductive wires 42, 44 may run along the length of the lumen 41 to deliver the electrical current that is necessary to create the shockwaves at the distal end 17 of the probe 10. The stiffening element 46 may also be placed within the lumen 41 of the probe 10. The stiffening element 46 may be secured to the body 40 of the probe 10 through a variety of methods, including but not limited to: adhesives. A variety of materials may be used for the stiffening element 46, including but not limited to: nitinol. The stiffening element 46 may provide extra support to the flexible body 40 of the probe 10, thereby making the probe 10 less likely to buckle or kink.

However, the addition of a stiffening element 46 may be accompanied by the sacrifice of probe 10 flexibility. The flexibility of the probe 10 is an important design consideration since the probe must be advanced through the twists and turns of the gastrointestinal tract and then deflected 90 degrees or more at the elevator of the duodenoscope 14. Therefore, it may be preferable to design the stiffening element 46 in such a way that minimizes the loss of flexibility while still providing the necessary added stiffness to prevent or limit kinking and buckling of the probe 10 during advancement through the cholangioscope 11. As shown in the graph in FIG. 3, the force required to advance the probe 10 through the working channel 13 of the cholangioscope 11 increases as the distal end 17 of the probe 10 advances further distally into the working channel 13. Further, as the force required to advance the probe increases, the need for a stiffening element 46 increases due to the increased likelihood of the exposed portion 34 buckling. Thus, the need for a stiffer stiffening element 46 that provides sufficient support to the probe 10 increases the further the probe 10 is distally advanced.

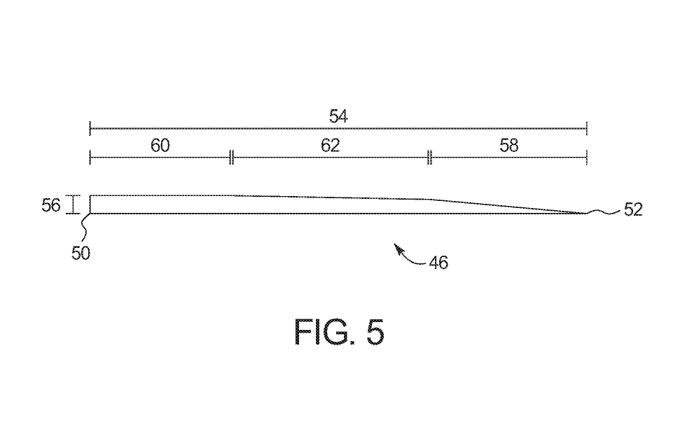

Therefore, it may be desirable to use a stiffening element 46 with a varying diameter or thickness along the length of the probe 10. For example, as shown in FIG. 5, a stiffening element 46 with a proximal end 50, distal end 52, and length 54 is shown. The stiffening element 46 further includes a distal portion 58, a proximal portion 60, and a central portion 62. As can be seen, the diameter 56 of the stiffening element 46 varies along the length 54 of the stiffening element 46. Specifically, the diameter 56 is at its largest diameter at the proximal end 50 and tapers down to its smallest diameter at the distal end 52. As the diameter 56 (or alternatively thickness) of the stiffening element 46 increases, the greater strength and support it provides to the probe 10. However, the flexibility of the probe 10 decreases as the diameter 56 of the stiffening element increases. Therefore, since maximum flexibility is desired for the probe 10 while still maintaining enough stiffness to resist kinking or buckling of the exposed portion 34 of the probe 10, the diameter 56 of the stiffening element 46 may be varied along the length of the probe 10 proportionally to the stiffness required to prevent kinking or buckling of the exposed portion 34. Therefore, since the probe 10 is at a low risk of kinking or buckling when the distalmost portion of the probe 10 is inserted into the working channel, the diameter 56 and proportional stiffness of the distal portion 58 of the stiffening element 46 may be small, therefore allowing the distalmost portion of the probe 10 to maintain maximum flexibility. However, since the exposed portion 34 of the probe 10 is at a high risk of buckling when the distal end 17 of the probe 10 is advanced past the deflection point 30, the diameter 56 and proportional stiffness of the proximal portion 60 of the stiffening element 46 may be large. The diameter 56 may slowly increase from the distal end 52 to the proximal end 50 in a manner proportional to the force required to advance the probe 10 distally.

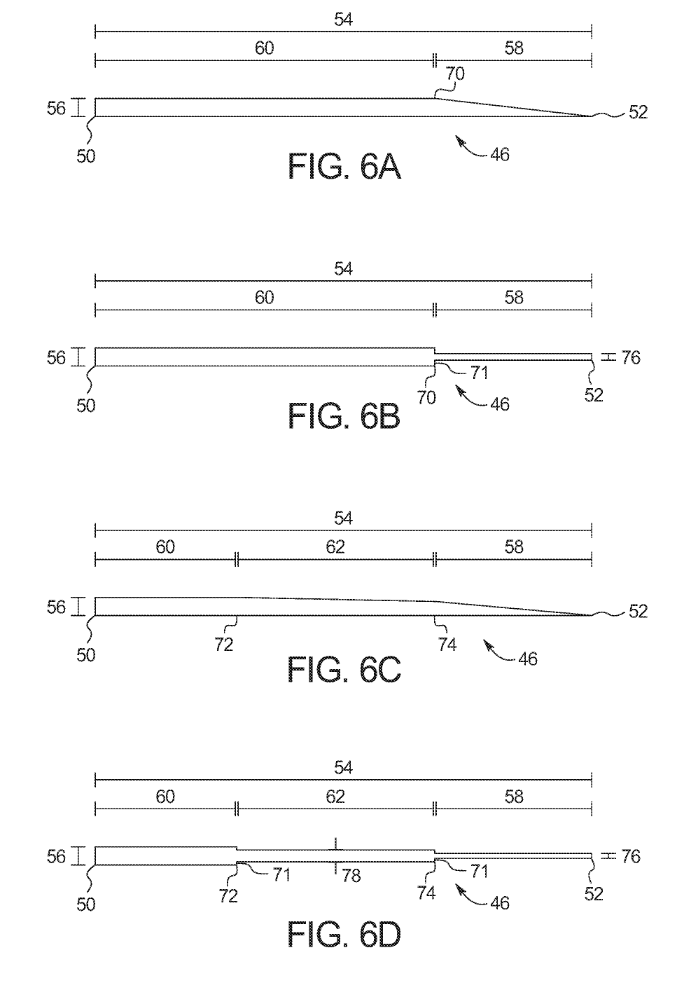

FIGS. 6A-6D show several more exemplary embodiments of the stiffening element 46. FIG. 6A shows a stiffening element 46 with a diameter 56, a proximal end 50, distal end 52, and a length 54 extending from the proximal 50 to distal end 52. The proximal portion 60 of the stiffening element 46 has a constant diameter 56. However, the distal portion 58 includes a taper from the transition point 70 (where the proximal portion 60 and distal portion 58 meet) to the distal end 52. In FIG. 6B, the diameter 56 of the proximal portion 60 is constant. The diameter 76 of distal portion 58 is constant, but is smaller than the diameter 56 of the proximal portion 60. A step 71 may be included at the transition point 70 between the proximal portion 60 and distal portion 58. FIG. 6C shows a stiffening element 46 with a proximal portion 60 having a constant diameter 56. The central portion 62 tapers from a larger diameter 56 at the first transition point 72 to a smaller diameter 56 at the second transition point 74. The distal portion 58 also tapers from a larger diameter 56 at the second transition point 74 to a smaller diameter 56 at the distal end 52. However, the rate of taper for the distal portion 58 is greater than the rate of taper for the central portion 62. Alternatively, the rate of taper for the central portion 62 may be greater than the rate of taper for the distal portion 58. FIG. 6D shows a stiffening element 56 with a proximal portion 60 having a constant diameter 56, a central portion 62 having a constant diameter 78 smaller than the constant diameter 56 of the proximal portion 60, and a distal portion 58 having a constant diameter 76 smaller than the constant diameter of the central portion 62. Steps 71 between the proximal portion 60 and central portion 62, and the central portion 62 and distal portion 58 are located at the first and second transition points 72, 74, respectively.

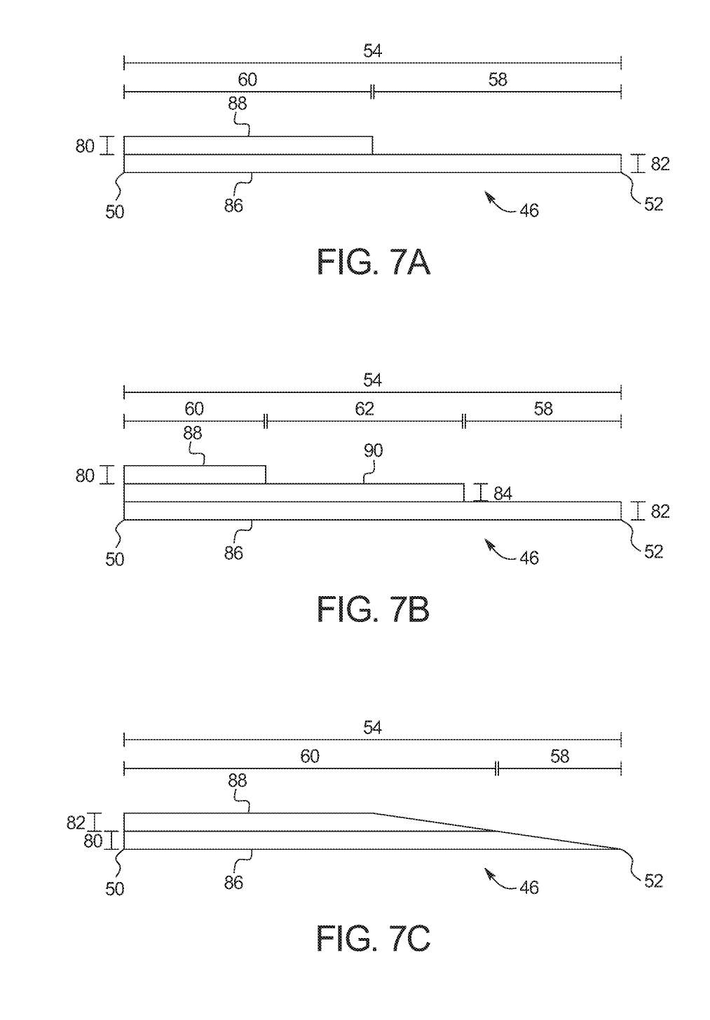

FIGS. 7A-7C show additional embodiments of the stiffening element 46. Specifically, the embodiments of FIGS. 7A-7C use multiple pieces of material to vary the stiffness of the stiffening element 46 instead of a single piece of material with a varying diameter. FIG. 7A shows a stiffening element 46 with a first stiffener 86 and a second stiffener 88. The first stiffener 86 extends from the proximal end 50 to the distal end 52 of the stiffening element 46 and may have a constant diameter 82. The second stiffener 88 extends through only a proximal portion 60 of the stiffening element 46 and may have a constant diameter 80. Since both the first and second stiffeners 86, 88 extend at least through the proximal portion 60 of the stiffening element 46 the proximal portion 60 has a greater stiffness than the distal portion 58. The first and second stiffeners 86, 88 may be attached together using a variety of methods, including but not limited to: adhesives. FIG. 7B shows a stiffening element 46 with a first stiffener 86, second stiffener 88, and third stiffener 90. The first stiffener 86 extends from the proximal end 50 to the distal end 52 of the stiffening element 46 and may have a constant diameter 82. The second stiffener 88 may have a constant diameter 80 and extends through the proximal portion 60 of the stiffening element 46. The third stiffener 90 may have a constant diameter 84 and extends through the proximal and central portions 60, 62 of the stiffening element 46. Since all three stiffeners 86, 88, 90 extend through the proximal portion 60, the proximal portion 60 has a high stiffness. The central portion 62 has a lower stiffness than the proximal portion 60, since only the first and third stiffeners 86, 90 extend through the central portion 62, and the distal portion 58 has the lowest stiffness since only the first stiffener 86 extends through the distal portion 58. FIG. 7C shows a stiffening element 46 with a first stiffener 86 and a second stiffener 88. The first stiffener 86 extends from the proximal end 50 to the distal end 52 of the stiffening element 46. The first stiffener 86 further includes a distally tapered diameter 80. The second stiffener 88 extends through the proximal portion 60 of the stiffening element 46 and has a distally tapered diameter 82. The above embodiments are just three examples of using multiple pieces of material for a stiffening element 46 with varying stiffness, additional variations are contemplated, including the use of more than three separate stiffeners 46, along with varying the materials or diameters used for each stiffener.

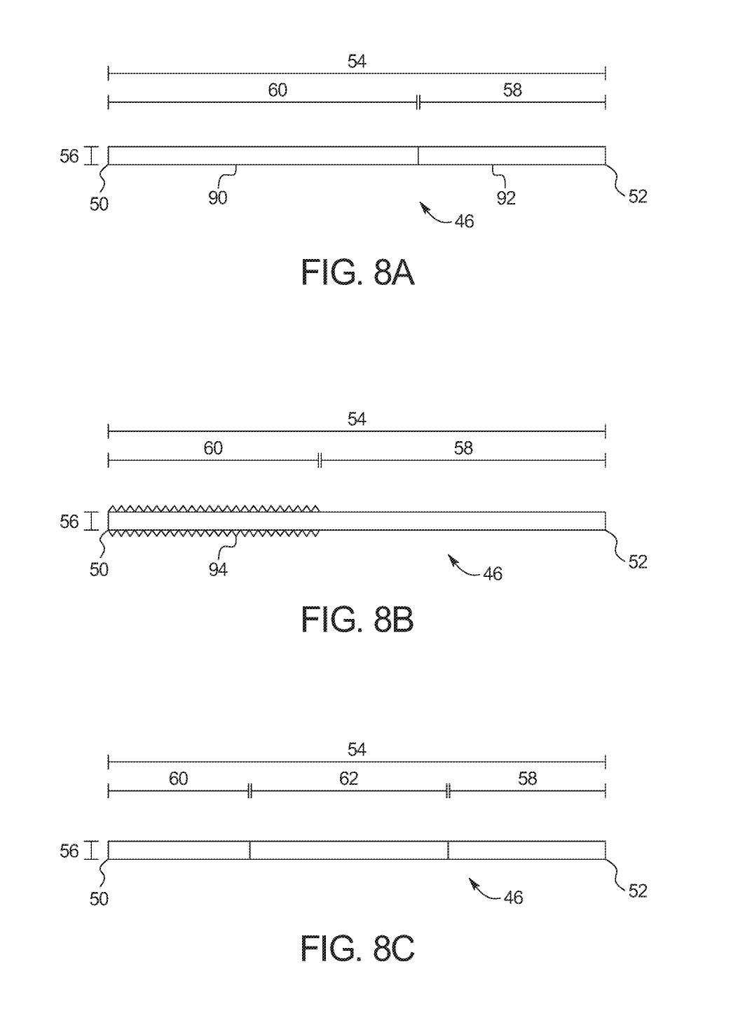

Further, rather than varying the thickness of the stiffening element 46 to vary the stiffness along the length 54 of the stiffening element 46, the material properties of the stiffening element 46 may be varied instead as shown in FIGS. 8A-8C. Therefore, the stiffening element 46 may have a constant outer diameter 56 with varied material properties that provide maximum stiffness at the proximal end 50 of the stiffening element 46 while providing minimum stiffness and maximum flexibility at the distal end 52 of the stiffening element 46. FIG. 8A shows a stiffening element 46 with a proximal portion 60 and a distal portion 58. The proximal portion 60 of the stiffening element 46 includes a first material 90 while the distal portion 58 of the stiffening element 46 includes a second material 92. The two materials 90, 92 may be attached together using a variety of methods. The materials 90, 92 may have varying stiffness properties, with the first material 90 having a greater stiffness than the second material 92. FIG. 8B shows a stiffening element 46 made of a single piece of material. The proximal portion 60 further includes a coating 94 that increases the stiffness of the proximal portion 60. Examples of coatings 94 include shrink tubing and other polymer wraps. FIG. 8C shows a stiffening element 46 made of a single piece of material. However, the portions 60, 62, and 58 may be heat treated in various ways to increase or decrease the stiffness of those portions 60, 62, 58. In one example, the proximal portion 60 may be heat treated to have a high stiffness, while the central portion 62 is heat treated to have a stiffness that is lower than the stiffness of the proximal portion 60. The distal portion 58 may remain untreated and have a lower stiffness than the proximal and central portions 60, 62. The above embodiments are just three examples of using multiple materials or heat treatment to vary the stiffness of the stiffening element 46. Additional variations on these embodiments are contemplated, including the use of more than two materials and heat treatment in combination with multiple materials.

While the above embodiments describe stiffening elements 46 that extend along the entire length of the probe 10, the stiffening element 46 may extend along only a portion of the probe 10. For example, the stiffening element 46 may extend from the proximal end of the probe 10 to a point proximal the distal end of the probe 10. Alternatively, multiple separate stiffening elements 46 may be used within a single probe 10, each with properties similar to the stiffening elements 46 described above. Further, various design features of the stiffening element 46 in the above mentioned embodiments may be mixed and matched with other embodiments as desired.

The probe 10 and stiffening element 46 may be used in a variety of applications with varying lengths and designs. In one example, the probe 10 may be around 200-300 centimeters in length. The length of the working channel 12 of the duodenoscope 14 may be about 140-160 centimeters and the length of the working channel 13 of the cholangioscope 11 may be around 200-250 centimeters. The proximal portion 60 of the stiffening element may be 15 centimeters in length or greater. It may be preferable for the distal portion 58 to range from 5-35 centimeters in length; however, it may extend as far as or greater than half the length of the working channel 13 of the cholangioscope 11. The central portion 62 may extend between the proximal and distal portions 60, 58, and may vary widely in length. In one example, the central portion 62 may range from 30-120 centimeters in length. These dimensions are merely exemplary, and the lengths of the probe 10, stiffening element 46, and the portions 58, 60, 62 of the stiffening element 46 may be further varied.

The embodiments described above show just several potential designs of a stiffening element. Many other stiffening elements with varying diameters or thicknesses may be used. Further, the stiffening element need not be cylindrical in shape with a circular cross section. For example, the stiffening element may have a rectangular, square, ovular, or other shaped cross-section. The cross-section of the stiffening elements may also include grooves configured to accommodate the conductive wires 42, 44, thus allowing for an overall reduction in the diameter of the probe 10. The grooves may also aid in preventing the conductive wires from contacting each other, thereby reducing the possibility of electrical shorting.

Additionally, while the stiffening elements 46 may be made of a metallic material, they may also be made of a non-metallic, or non-conductive material. Non-conductive stiffening elements 46 may be desirable to prevent or limit the risk of shorting the conductive wires. Similarly, the stiffening elements 46 may instead or also be coated or wrapped in a non-metallic material to limit the risk of shorting the conductive wires.

While the present disclosure describes the embodiments in terms of a lithotripsy probe used during a biliary procedure, the stiffening element 46 may be used in any lithotripsy procedure to limit kinking and buckling of the probe when inserted into a patient. Further, the anti-kinking and buckling improvements may be used with a variety of other medical devices unrelated to lithotripsy, such as catheters used in a variety of medical procedures. Also, the improvements described above may be used in a variety of non-medical applications.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A lithotripsy probe, comprising:
an elongate body comprising a proximal end, a distal end, and a lumen extending therethrough; and
a stiffening element comprising a proximal end, a distal end, and a length extending from the proximal end to the distal end, the stiffening element disposed within the lumen of the elongate body;
wherein the stiffening element further comprises a stiffness, the stiffness varying along the length of the stiffening element, wherein the stiffness of the proximal end of the stiffening element is greater than the stiffness of the distal end of the stiffening element.

2. The lithotripsy probe of claim 1, further comprising:
first and second conductive wires extending through the lumen of the elongate body, the first and second conductive wires configured to deliver electrical energy to the distal end of the elongate body.

3. The lithotripsy probe of claim 1, wherein:
the stiffness of the stiffening element increases along the length of the stiffening element from a lower stiffness at the distal end of the stiffening element to a greater stiffness at the proximal end of the stiffening element.

4. The lithotripsy probe of claim 1, wherein:
the proximal end of the stiffening element is substantially coterminous with the proximal end of the elongate body and the distal end of the stiffening element is substantially coterminous with the distal end of the elongate body.

5. The lithotripsy probe of claim 1, wherein:
the proximal end of the stiffening element is substantially coterminous with the proximal end of the elongate body and the distal end of the stiffening element is proximal the distal end of the elongate body.

6. The lithotripsy probe of claim 1, wherein:
the stiffening element further comprises a proximal portion and a distal portion, the proximal portion comprising a substantially constant outer diameter and the distal portion comprising an outer diameter with a distally decreasing taper.

7. The lithotripsy probe of claim 1, wherein:
the stiffening element further comprises a proximal portion and a distal portion, the proximal portion comprising a substantially constant outer diameter and the distal portion comprising a substantially constant outer diameter that is smaller than the outer diameter of the proximal portion, the stiffening element further comprising a step at a transition point between the proximal and distal portions.

8. The lithotripsy probe of claim 1, wherein:
the stiffening element further comprises a proximal portion, a central portion, and a distal portion, the proximal portion comprising a substantially constant outer diameter, the central portion comprising a substantially constant outer diameter that is smaller than the outer diameter of the proximal portion, the distal portion comprising a substantially constant outer diameter that is smaller than the outer diameter of the central portion; and
the stiffening element further comprises a first step at a first transition point between the proximal and central portions and a second step at a second transition point between the central and distal portions.

9. The lithotripsy probe of claim 1, wherein:
the stiffening element further comprises a proximal portion, a distal portion, and a first and second stiffener, the first stiffener extending from the proximal end of the stiffening element to the distal end of the stiffening element, the second stiffener extending through the proximal portion of the stiffening element.

10. The lithotripsy probe of claim 9, wherein:
the stiffening element further comprises a central portion and a third stiffener, the third stiffener extending through the proximal and central portions of the stiffening element.

11. The lithotripsy probe of claim 9, wherein:
the first stiffener comprises a distally directed taper from a larger proximal diameter to a smaller distal diameter; and
the second stiffener comprises a distally directed taper from a larger proximal diameter to a smaller distal diameter.

12. The lithotripsy probe of claim 1, wherein:
the stiffening element further comprises a proximal portion comprising a first material and a distal portion comprising a second material, the first material having a greater stiffness than the second material.

13. The lithotripsy probe of claim 12, wherein:
the stiffening element further comprises a central portion comprising a third material, the third material having a stiffness that is greater than the stiffness of the second material, but is less than the stiffness of the first material.

14. The lithotripsy probe of claim 1, wherein:
the stiffening element further comprises a proximal portion and a distal portion, the proximal portion comprising a coating that increases the stiffness of the proximal portion.

15. The lithotripsy probe of claim 1, wherein:
the stiffening element further comprises a proximal portion and a distal portion, the proximal portion being heat treated to increase the stiffness of the proximal portion, the proximal portion having a stiffness that is greater than the stiffness of the distal portion.

16. The lithotripsy probe of claim 1, wherein:
the stiffening element further comprises a diameter, the diameter increasing at a constant rate from a smaller diameter at the distal end of the stiffening element to a larger diameter at the proximal end of the stiffening element.

17. The lithotripsy probe of claim 1, wherein:
a diameter of the stiffening element is greater at the proximal end of the stiffening element than at the distal end of the stiffening element.

18. A lithotripsy kit, comprising:
a scope comprising a proximal end, a distal end, and a working channel extending therethrough, the scope further comprising a proximal entrance to the working channel; and
a probe comprising an elongate body comprising a proximal end, a distal end, and a lumen extending therethrough, the probe further comprising a stiffening element comprising a proximal end, a distal end, and a length extending from the proximal end to the distal end, the stiffening element disposed within the lumen of the elongate body;
wherein the probe is advancable through the working channel of the scope, the stiffening element comprising an adjacent point that is adjacent to the proximal entrance of the working channel, the adjacent point varying in position along the length of the stiffening element as the probe is distally advanced through the working channel, the probe further comprising a force required to advance the probe through the working channel of the scope, the force increasing as the adjacent point moves proximally along the length of the stiffening element;
wherein the stiffening element further comprises a stiffness that varies along the length of the stiffening element, the stiffness increasing along the length of the stiffening element from the distal end to the proximal end, wherein the stiffness of the stiffening element at the adjacent point is proportional to the instant force required to advance the probe through the working channel of the scope at the adjacent point's current position along the length of the stiffening element.
